(19) United States
(12) Patent Application Publication
(10) Pub. No.: US 2021/0346192 A1
(43) Pub. Date: Nov. 11, 2021

(54) SYSTEMS AND METHODS OF GENERATING COLD SLURRY FOR INJECTION

(71) Applicant: MIRAKI INNOVATION THINK TANK LLC, Cambridge, MA (US)

(72) Inventors: Christopher VELIS, Lexington, MA (US); Karen MILLER, South Dartmouth, MA (US); Rainuka GUPTA, Newton, MA (US); William Roger MAINWARING-BURTON, Cambridge, MA (US); Bradley Leo GUERTIN, Roseville, MN (US); Avi Aaron KURLANTZICK, Dedham, MA (US); Andrew Arthur DAVENPORT, Englewood, CO (US); Nicholas Robert TOSTA, Boston, MA (US); Bryan Ellis WAGENKNECHT, West Roxbury, MA (US)

(73) Assignee: MIRAKI INNOVATION THINK TANK LLC, Cambridge, MA (US)

(21) Appl. No.: 17/283,685

(22) PCT Filed: Oct. 10, 2019
(86) PCT No.: PCT/US2019/055634

§ 371 Date: Apr. 8, 2021

Related U.S. Application Data
(60) Provisional application No. 62/743,908, filed on Oct. 10, 2018.

(51) Int. Cl. A61F 7/00; A61F 7/12

(52) U.S. Cl. CPC A61F 7/0085; A61F 2007/0063; A61F 2007/126; A61F 7/12

(57) ABSTRACT

Systems and methods for generating slurry for injection are provided. Systems comprise a container and a device. The container comprises a solution for administration via an injection needle of a predetermined size. The device is capable of receiving the solution and producing a slurry having ice particles capable of flowing through the injection needle. Methods comprise selecting a container having the solution, receiving the solution in the device, and producing, in the device, the slurry having ice particles capable of flowing through the injection needle.

### TECHNICAL FIELD

The invention is directed to systems and methods of generating slurry for injection.

### BACKGROUND

Subcutaneous fat is found just beneath the skin, helps store energy for the body, and provides a layer of protection for internal organs of humans. However, abnormal or excessive fat accumulation impairs health and leads to being overweight or obese. Humans that are overweight or obese have an increased risk of death and suffer from several medical and cosmetic issues.

One method of treating humans suffering from medical and cosmetic issues related to excess fat is removal of the excess fat. Conventional surgical methods of fat removal, such as liposuction, are invasive, painful, time-consuming, and require surgery and follow-up visits to a healthcare facility. Topical cryolipolysis refers to devices that are placed on the skin to remove subcutaneous fat for aesthetic purposes by treating the tissue with cool temperatures to selectively target fat cells. However, these treatments also suffer from drawbacks including treatment times that are longer and colder than needed to selectively target fat, limited treatment areas due to standard applicator use, lack of precision, and limits to the depth and amount of fat that can be removed.

Therefore, it is desirable to provide improved cryolipolysis systems and methods.

### SUMMARY

The present invention provides systems and methods for generating slurry for administration to a subject. The slurry of the present invention can be used in selective injection cryolipolysis for fat removal, selective targeting of non-adipocyte, lipid rich tissue, and connective tissue remodeling, while avoiding non-specific hypertonic injury to tissue. For example, a slurry can be administered (e.g., injected) to a subject such as a human subject to selectively remove fat cells. The present invention provides for customizable slurry generation where a slurry is generated having certain properties based on the patient and/or treatment.

The invention provides containers comprising one or more solution ingredients that generate a slurry having the desired properties. In embodiments of the invention, the one or more containers comprise one or more ingredients of the solution. In other embodiments of the invention, each ingredient of the solution has a separate container. The invention allows healthcare providers to tailor the slurry to the application at hand. For example, the invention allows for ease of use for healthcare providers, as the healthcare providers merely have to select the container comprising a solution that corresponds to a particular needle size or a particular treatment area.

A solution is used to generate slurry having certain desired properties such as the desired needle gauge to be used for a procedure, the slurry ice coefficient (defined as the percentage of ice in the slurry), the flowability of the slurry through the delivery device such as a needle, the tonicity of the slurry, the temperature of the slurry and volume of slurry needed for a treatment. As an example, more fat cells may be eliminated when a slurry having a higher ice coefficient is applied to the targeted area. Ice particle size/shape in the slurry affects the size of the cannula such as a needle that may be used. For example, the needle must be large enough to fit the ice particles flowing through without the ice particles clogging or blocking the needle. In order to achieve a slurry that has a high percentage of ice, but can also be injected through a smaller needle, it is desirable to create small globular ice crystals at the appropriate ice coefficient.

Administration of slurries for fat removal varies for different patients, as different patients have different needs. For example, one patient may be overweight and require a large volume of slurry for treatment. The patient may also have thick skin. Such a patient may require use of an injection needle having a larger gauge size than other patients may require. For instance, the overweight patient with thick skin may require a needle having a gauge size of about 12G in order to pierce the skin and deliver an effective amount of slurry to the desired tissue. As another example, a patient may be interested in slurry treatment for fat removal in an area of the body such as the face where a smaller gauge needle may be desirable. In such an example, a needle having a size of about 21G may be used to deliver the slurry by injection.

Certain aspects of the invention are directed to methods of generating a slurry in which one or more ingredients are introduced to a slurry generator and parameters of the slurry generator are adjusted, resulting in a slurry comprising certain properties. In some embodiments, the parameters are shown on a display, which may be an interactive display, that is part of, or associated with, the slurry generator. Non-limiting examples of such parameters are temperature, flow rate, ice percentage by volume, and slurry generation time.

Certain aspects of the invention are directed to systems for generating slurry in which the system comprises one or more containers, each comprising one or more ingredients, and a slurry generator. In either case, the slurry is configured to be introduced to a patient. The container or containers is/are inserted into the slurry generator. In some instances, each of the one or more solution ingredients is in a separate container. In some embodiments, each separate container is in fluid communication with the slurry generator. Various systems and methods for generating slurry can be employed. For example, slurry can be generated using a continuous flow system, an agitated system or a hybrid system (both continuous flow and agitation). In a continuous flow or hybrid system, the flow rate of the slurry flowing through the slurry generator should be selected to maintain flow and generate ice particles consistently throughout the slurry.

The slurry is produced by adjusting the one or more solution ingredients provided to the slurry generator. The solution ingredients comprise liquid water and one or more additives. In some embodiments, the one or more additives are additives affecting flowability and/or tonicity of the slurry. Any suitable additive may be added to the solution, including any substance on the FDA GRAS list.

Therefore, embodiments of the invention are directed to a stable, repeatable method of providing a solution capable of generating slurry having certain properties, for example, ice particles having a particle size capable of flowing through a surgical needle of a predetermined size.

### BRIEF DESCRIPTION OF THE DRAWINGS

FIG. 1 shows an embodiment of the invention including a block diagram of a system for generating a slurry.

FIG. 2 shows another embodiment of the invention including a block diagram of a system for generating a slurry.

FIG. 3 shows an exemplary slurry generator according to the present invention.

### DETAILED DESCRIPTION

The present invention is directed to systems and methods for generating a slurry using a customizable, stable, and repeatable method. Systems and methods of the invention are used to generate a volume of a slurry having one or more desired properties, for example an ice particle size/shape capable of flowing through a cannula. In some instances, the cannula is a needle, such as a surgical needle of a predetermined size. The slurry can be administered to a subject such as a human through an injection by the cannula or needle.

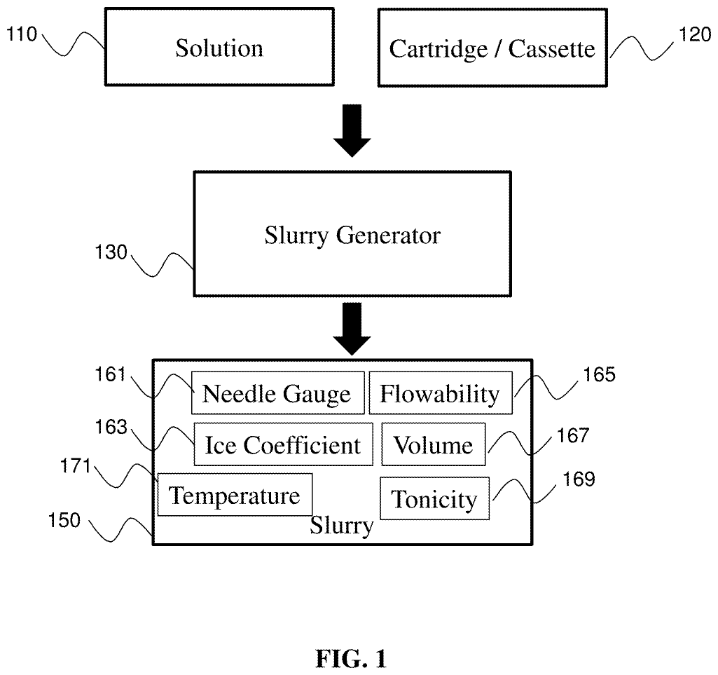

FIG. 1 shows an embodiment of the invention. Systems of the invention generate slurry by introduction of solution to a slurry generator 130. Optionally, the solution 110 may be provided in a cartridge or cassette 120. Outputs from the slurry generator 130 include the slurry 150. The slurry 150 is generated with certain desired characteristics or properties, such as ice particles sized/shaped to flow through a desired needle gauge 161, ice coefficient 163, flowability 165, volume 167 tonicity 169 and temperature 171.

Varying or adjusting inputs to the slurry generator achieves a desired output of slurry. By adjusting the solution input to the slurry generator, the invention allows for customized slurry for various applications. For example, solution ingredients and their respective amounts (volume and/or concentrations) can be selected and added to the generator to generate a slurry having certain desired properties, for example, a specified ice coefficient.

Different patients and/or treatment areas may require slurry having certain properties. For example, a patient that has thick skin and a high amount of excess fat may require a higher volume of slurry and a larger needle size in order for the needle to pierce through the skin and inject slurry to the desired tissue. A needle having a gauge size of about 12G may be used for such a patient. Conversely, a patient with little excess fat may require a small needle size. Additionally, a smaller needle size reduces the risk of scarring and anxiety associated with large needle use. A needle having a gauge size of about 21G may be used for such a patient. As another example, it may be desirable to use a smaller needle and/or lower volume of slurry for a smaller treatment area such as a chin whereas a larger needed and/or volume of slurry may be desirable to treat an abdomen. When a larger gauge size is selected, larger ice particles may be generated in the slurry.

### Inputs

Inputs to the system can include solution, solution ingredients, cartridge 120, and user inputs to the slurry generator.

### Solution/Solution Ingredients

By adjusting the solution input to the slurry generator, the invention allows for customized slurry for various applications. In certain embodiments, a solution for making a slurry comprises liquid water and one or more additives affecting the various properties of the slurry. For example, agents affecting viscosity can affect the flowability of the slurry (relates to ice particles capable of flowing through a cannula). Additionally, additives that affect tonicity can alleviate adverse inflammatory and other effects at the injection site. Any acceptable or suitable volume and/or concentration of water and one or more additives may be used in the present invention and may be selected based on the desired outcome which can be based on the patient and/or treatment. Solution compositions are described in international PCT Application Serial Number PCT/US19/54828 which is incorporated by reference in its entirety herein.

Examples of agents affecting viscosity include celluloses (i.e. carboxymethylcellulose, hydroxyethylcellulose, hydroxypropylmethylcellulose, methylcellulose), polyvinyl alcohol, polyvinylpyrrolidone, xanthan gum, polyethylene glycol, guar gum, locust bean gum, carrageenan, alginic acid, gelatin, acacia, and carbopol.

Examples of additives affecting tonicity include salts, cations, anions, polyatomic cations, polyatomic anions, sugars, and sugar alcohols. Increased levels of agents affecting tonicity (otherwise known as osmotically active compounds) enable the production of small, globular, injectable ice crystals that are able to pass through a needle without clogging. However, the increased levels of agents affecting tonicity can result in hypertonic injury to tissue, as once they are injected into the body, the high osmolality of the slurry can dehydrate adjacent tissue, induce non-specific cell damage. In some embodiments, additives are inactive ingredients.

In some embodiments, the additives comprise one or more of a salt, a sugar, and a thickener. Any suitable additive may be added to the solution or the slurry, including any substance on the FDA GRAS list, which is incorporated herein in its entirety.

Any acceptable or suitable volume and/or concentration of one or more additives may be used and may be selected based on the treatment. For example, for intradermal, subcutaneous, or intramuscular routes of administration, additives include sodium chloride (saline), glycerin/glycerol, dextrose, sodium CMC, xanthan gum, and polyethylene glycol. For example, acceptable concentrations of sodium chloride (saline) are about 0.9% for soft tissue use and about 2.25% for subcutaneous use, while acceptable concentrations of glycerin/glycerol are about 1.6% to about 2.0% for dermal use and about 15% for subcutaneous use. Further, acceptable concentrations of dextrose are about 5% w/v for intramuscular use and about 7.5% per unit dose for intramuscular-subcutaneous use. For example, acceptable concentrations of sodium CMC are about 0.75% for intralesional use, about 3% for intramuscular use, and about 0.5% to about 0.75% for soft tissue use. As another example, acceptable concentrations of xanthan gum are about 1% for intra-articular use in animal studies and about 0.6% for FDA ophthalmic use. Further, acceptable concentrations of polyethylene glycol, such as Polyethylene Glycol 3350, are about 2.0% to about 3.0% for FDA soft tissue use and about 4.42% for subcutaneous use.

In some embodiments, the salt is one or more of sodium chloride, potassium, calcium, magnesium, hydrogen phosphate, hydrogen carbonate. In some embodiments, glycerol is an additive. In some embodiments, dextrose is an additive. In some embodiments, additives for affecting the viscosity include CMC and Xanthan Gum. In some embodiments, an additive may comprise a buffer to stabilize the pH. In some embodiments, an additive may comprise an emulsifier to create a smooth texture. In some embodiments, an additive may comprise a nanoparticle, for example, TiO2. The smaller sized particles in the solution may increase the number of nucleation sites, thus enabling creation of smaller ice crystals. In some embodiments, an additive may comprise an agent configured as coating for the ice crystals which may prevent agglomeration after formation may be included. In some embodiments, an additive may comprise IVF Synthetic Colloids at amounts of about 6.0% Hetastarch in about 0.9% sodium chloride, at about 309 mOsm; Poloxamer 188 at amounts of about 0.2% subcutaneous; Propylene Glycol at amounts of about 0.47% to about 1.4%; Benzyl Alcohol at amounts of FDA about 0.9% to about 1.4%; gelatin at amounts of FDA subcutaneous about 16%; and Icodextrin used frequently in peritoneal dialysis at amounts of about 7.5%.

In certain embodiments, the solution has an osmolarity lower than about 2,200 mOsm/L. In some embodiments, the osmolarity is less than about 600 mOsm/L. In such an embodiment, the slurry may comprise about 0.9% saline; about 1.0% to about 2.0% dextrose; about 1.0% to about 1.6% glycerol; less than about 0.5% sodium CMC; and less than about 0.6% xanthan gum. In one embodiment, the slurry composition may be about 500 mOsm/kg to about 700 mOsm/kg and comprise about 0.9% to about 1.4% saline; about 2.0% to about 4.0% dextrose; about 1.7% to about 2.0% glycerol; about 0.6% to about 1.0% sodium CMC; and about 0.6% to about 1.0% xanthan gum. In another embodiment, the slurry composition may be about 700 mOsm/kg to about 900 mOsm/kg and comprise about 1.5% to about 1.7% saline; about 5.0% to about 7.5% dextrose; about 3.0% to about 5.0% glycerol; about 1.0% to about 3.0% sodium CMC; and about 1.0% xanthan gum. In some embodiments, the slurry composition may be greater than about 1,000 mOsm/kg. In such an embodiment, the slurry may comprise about 1.8% to about 3.0% saline; about 10% dextrose; greater than about 5.0% glycerol; sodium CMC; and xanthan gum.

Any of the above solution ingredients and their respective amounts (volume and/or concentrations) can be selected and added to the slurry generator to generate a slurry having certain properties.

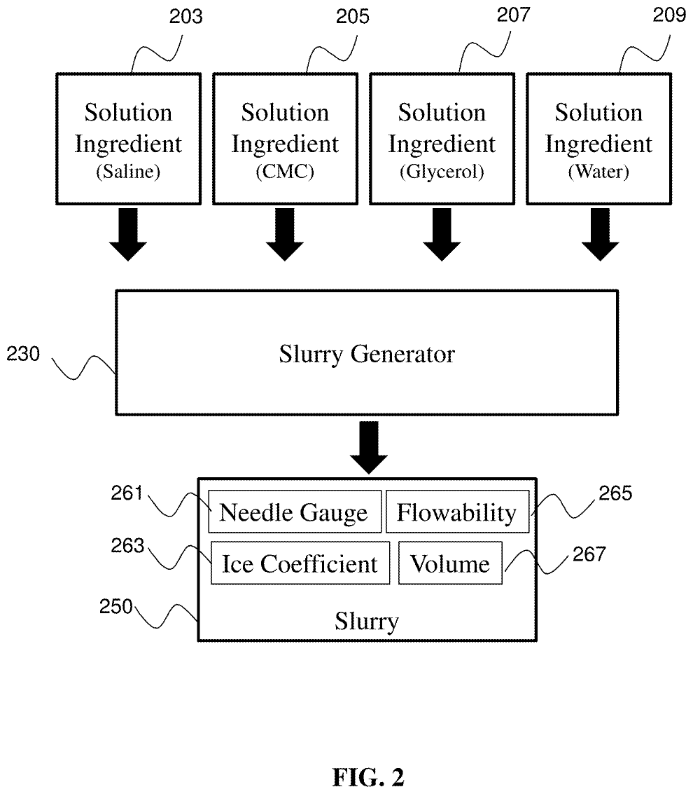

FIG. 2 illustrates an exemplary system of the present invention comprising four containers comprising solution ingredients 203, 205, 207 and 209, a slurry generator 230 and slurry 250 having certain properties, 261, 263, 265, and 267. In this example, the solution ingredients include water 209 and additives including saline 203, glycerol 207 and CMC 205 at desired concentrations. A user can customize a solution using these four ingredients based on the desired properties of the slurry. In some embodiments, the solution can comprise NaCl at about 2.25% by mass or lower, glycerol at about 2% by mass or lower, and CMC at about 0.75% by mass or lower. Additional additives (not shown) may be included to affect various properties of the slurry.

### Cartridge

The cartridge 120 can be configured and provided to slurry generator to generate slurry having certain properties. In some embodiments, the cartridge 120 comprises tubing for the solution to flow within the cartridge 120, materials and thermal properties, each of which can be selected to generate a slurry having certain properties.

### User Inputs

A user can set and adjust various input and/or output parameters such as volume, needle gauge, and treatment area to the system to generate slurry having certain properties.

### Slurry Generator

The slurry generator generates the slurry upon providing a solution. Various systems and methods for generating slurry can be employed. For example, slurry can be generated using a continuous flow system, an agitated system or a hybrid system (including both continuous flow and agitation). The slurry may be generated and customized by the type of system used and by adjusting the process parameters of the system. Example parameters include cooling temperature, solution/slurry flow rate, slurry generation time, agitator speed, gas flow rate, maintenance temperature, ice growth rate, and nucleation sites.

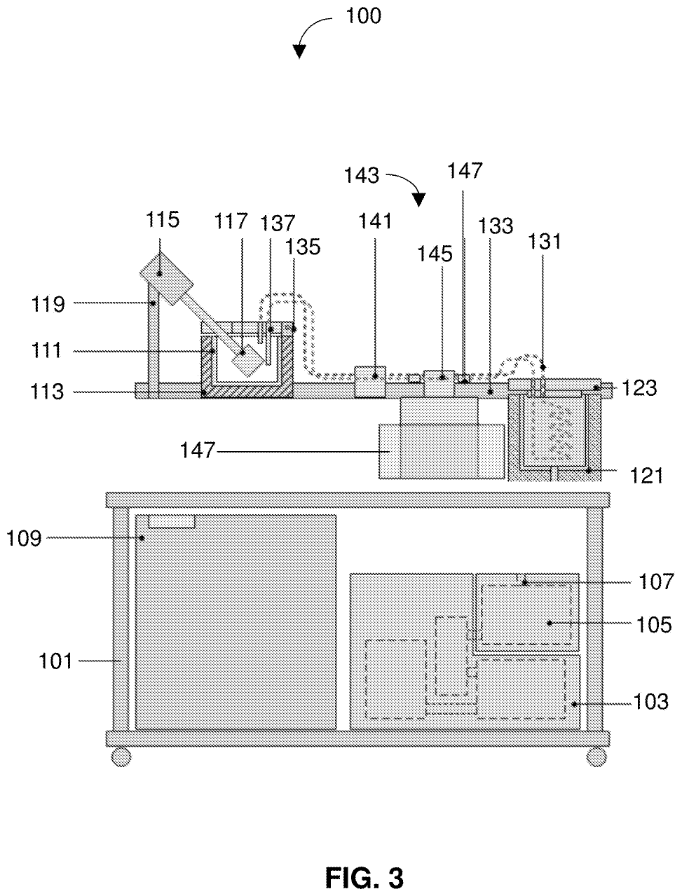

FIG. 3 shows an exemplary embodiment of a hybrid system 100 for generating a slurry. System 100 includes a base station 101 with a slurry reservoir 111 and a cooling device 103. Base station 101 may optionally include refrigerator 109, which may be used to contain pre-prepared solution, constituents of a solution, syringes for injection, thermal jackets for the syringes, and other components that may be used with system 100. When preparing a slurry, a solution used to generate the slurry may be introduced to slurry reservoir 111 and cooled by cooling device 103. As shown, cooling device 103 includes coolant reservoir 105, coolant opening 107, coolant insulation 121, and coolant cover 123. Although the connection is not shown, coolant reservoir 105 is in fluid connection with the portion of coolant reservoir 105 covered by coolant cover 123 and insulated by coolant insulation 121.

System 100 further includes circulation system 143, which includes a pump 145 in fluid communication with the slurry reservoir 111 via tubing 131 for circulating the solution at least from the slurry reservoir 111 to the cooling device 103. The pump 145 may be a peristaltic pump or any other suitable pump that moves the solution or slurry to and from coolant reservoir 105. Tubing 131 may be insulated by tubing insulation 133 to decrease the introduction of heat into the slurry while circulated by circulating system 143.

In this embodiment, insulation 113 is at least partially in contact with slurry reservoir 111. As shown, slurry reservoir 111 is covered by lid 135 with tubing connections 137 to connect slurry reservoir 111 to tubing 131 such that the slurry is in fluid communication with circulating system 143. Lid 135 may house agitator paddle 117. Agitator paddle 117 is connected to and driven by agitator motor 115. Agitator motor 115 may be supported by an agitator support 119. Agitator paddle 117 may agitate the solution or slurry while the slurry is being generated, while the slurry is being maintained, and/or after the slurry is prepared. By agitating the slurry, temperature may be more readily maintained throughout the volume, and agglomeration of ice particles may be reduced as well as stratification of the slurry. A more consistent slurry may be provided by agitation of the slurry as compared to a system lacking agitation.

System 100 optionally includes nucleator 141 and fluidic connectors 147 for connecting tubing 131 to pump 145. Nucleator 141 is connected to circulating system 143 and may induce nucleation in the solution such that ice particle generation is initiated. Upon nucleation, the circulating system may maintain a continuous flow of the slurry at least from the reservoir to the cooling device. This continuous flow throughout the system helps maintain a consistent temperature of the slurry, which improves the ice coefficient of the slurry, ice particle size, flowability, and effectiveness when administered. Accordingly, a more consistent slurry may be maintained throughout system 100, resulting in a substantial volume of slurry being ready for a treatment. For example, in a treatment involving four separate injections in separate abdominal positions, any variation between the first and last injection may be minimized by the continuous flow. In some embodiments, nucleation occurs spontaneously upon the system/solution reaching a particular temperature.

When generating a slurry, a solution used for generating a slurry may be provided to slurry reservoir 111. In alternative embodiments, components of the solution may be provided to or mixed in slurry reservoir 111. Circulating system 143 may then circulate the solution to and from cooling device 103 via tubing 131 and pump 145. As the solution circulates to and from coolant reservoir 105, which contains coolant that is colder than a temperature of the initial solution, heat from the solution may dissipate into the coolant through the tubing 131 and thereby cool the solution.

Once the solution is cooled to a certain temperature, nucleation may be induced by the nucleator 141 to form ice particles and generate the slurry. In some embodiments, nucleation is spontaneous. Nucleation is the first step in the formation of either a new thermodynamic phase or a new structure, such as by self-assembly or self-organization of ice particles in a solution containing water. Without a nucleation event, generation of the slurry may take longer and may result in an inconsistent slurry that lacks an appropriate ice coefficient, ice particle size, flowability, and effectiveness when administered.

However, nucleation may be induced by various physical, chemical, or other suitable methods. In one example, nucleator 141 may be a mechanical means of inducing nucleation. For example, nucleator 141 may be a pinch valve, in which an operator or a motorized unit may pinch at least a portion of tubing 131 to induce nucleation. In certain embodiments, a collapsible member may be used to induce nucleation, in which a force may be applied to the collapsible member to cause at least a part of it to collapse and thereby simulate a pinching motion and induce nucleation. For example, the collapsible member may be tubing or may have an elongated body of any suitable shape, such as a bulb shape, an elongated bulb shape, a tubular shape, or the like. In this example, a collapsible member in fluid communication with tubing 131, through which the solution or slurry is circulated, may pinch tubing 131 to cause nucleation. A force such as a mechanical force from a motor or a vacuum force may be applied to the collapsible member to cause at least a part of it to collapse.

In various embodiments, a cartridge (not shown) may be attached in fluid communication with the circulating system 143 or slurry reservoir 111 and receive a volume of slurry. The cartridge may then be used to administer the slurry to a subject through a cannula. For example, a cartridge may receive a volume of between 10 mL and 100 mL of slurry and be attached to a handheld unit with a needle of gauge size 18G. The slurry may then be administered to a subject through the needle. Various cartridges may be used with system 100 and the cartridges may be reuseable or disposable after a single use or more than one use. In one embodiment, the cartridge includes nucleator 141 and the continuous flow of slurry throughout circulating system 143 includes circulation through the cartridge and nucleator 141 within the cartridge. In another embodiment, the nucleator 141 is not within a cartridge and the cartridge simply receives a volume of slurry that may be injected. In one example, the cartridge may include an agitator to prevent agglomeration, reduce temperature differences within the volume, and maintain a consistent slurry through injection.

The generator further allows for stability of the properties of the slurry for the duration of treatment. For example, if treatment time is approximately one hour, the slurry should be stable for longer than one hour.

The solution/slurry flow rate through the system is another parameter that may be selected and adjusted. In some embodiments, the flow rate comprises about 20 ml/min to about 200 ml/min.

The temperature of the generator should be a temperature cold enough to generate ice particles but warm enough to avoid creating too much ice and clogging or blocking the flow of the slurry.

The slurry generation time is another device parameter that may be adjusted. The treatment time and time leading up to treatment with the slurry may be variable per patient situation. The slurry generation time may be any suitable slurry generation time. For example, the slurry generation time may be less than about 10 minutes to about 12 hours. In some examples, a patient may make a last-minute appointment or be a walk-in patient. In such cases, a healthcare professional may desire a quick slurry generation time, such as less than about 10 minutes. Other times, a healthcare professional may know that a patient is scheduled for an appointment first thing in the morning. In such cases, the healthcare professional may want to set a longer slurry generation time in order to prepare the slurry overnight so that the slurry is generated and ready to administer for the early morning appointment. Therefore, the healthcare professional may set a slurry generation time of about 12 hours.

### Outputs

The output from the slurry generator is slurry configured to be introduced to a patient. The slurry is generated with certain desired characteristics or properties, such as ice particles sized/shaped to flow through a desired needle gauge, flowability, ice coefficient, temperature, tonicity and volume of slurry generated. In some embodiments, the slurry comprises liquid water, ice comprising from about 2% to about 70% by volume, and one or more additives affecting one or more properties of the slurry.

In certain aspects of the invention, the solution can be tailored to allow generation of a slurry with a desired ice content and particle size. Factors considered in tailoring the solution include flowability, crystal size and morphology, ice percentage, ice temperature, maximum additive content by mass, volume of slurry produced, and preparation time and stability factors. In some embodiments, to allow flow through a distribution or aspiration system, the maximum ice particle size, or maximum crystal size, may be less than about 333 um in certain embodiments. Further, different slurry particle size distribution medium values (D50), or crystal size median values, may be achieved, such as between about 50 um and about 300 um. The standard deviation of the particle size distribution, or the crystal size variation, may stay constant for different median crystal size values. With respect to the crystal morphology, it is preferable that the crystals may be generally rounded in order to enable flow through any distribution or aspiration system. Dendritic ice, by its nature, may cause bridging and clogging in the delivery system and is therefore less desirable.

Flowability is a characteristic that determines the flow of ice particles capable of flowing through a delivery device. For example, the slurry may be injected through a cannula. In some embodiments, the cannula is a needle. In some embodiments, each ice particle has a particle size of less than about 1 mm. In some instances, the particle size is less than about 0.25 mm. Further, particle size of the ice is important when choosing the gauge size of a needle. A slurry can be generated for use with a needle having a gauge size ranging from about 8G to about 25G. Flowability of the ice may be determined by any suitable method. For example, one representative method of measuring flowability is to attempt to push ice through a needle. Comparison using the smallest needle size (or highest needle gauge) gives a comparative value for the flowability of the slurry.

Table 1 shows a chart having exemplary needle gauges and the corresponding inner diameter of each respective needle (in millimeters). In an embodiment of the invention, Table 1 can correspond to an identification number for the solution that will produce ice particles capable of flowing through a needle having the respective gauge size.

TABLE 1
Needle Gauge and Corresponding Inner Diameter Size

| Needle | Inner Diameter of |
|---|---|
| Gauge | Needle (mm) |
| 6 | 4.39 |
| 7 | 3.81 |
| 8 | 3.43 |
| 9 | 3 |
| 10 | 2.69 |
| 11 | 2.39 |
| 12 | 2.16 |
| 13 | 1.8 |
| 14 | 1.6 |
| 15 | 1.37 |
| 16 | 1.19 |
| 17 | 1.07 |
| 18 | 0.84 |
| 19 | 0.69 |
| 20 | 0.6 |
| 21 | 0.51 |
| 22 | 0.41 |
| 23 | 0.34 |
| 24 | 0.31 |
| 25 | 0.26 |
| 26 | 0.26 |
| 27 | 0.21 |
| 28 | 0.18 |
| 29 | 0.18 |
| 30 | 0.16 |
| 31 | 0.12 |
| 32 | 0.11 |
| 33 | 0.11 |
| 34 | 0.08 |

Inner Diameter (ID) for needle gauge sizes obtained from MilliporeSigma website (Sigma-Aldrich Corp., St. Louis, MO, USA).

Once the size of needle to be used for injection is decided, which may be on-demand, the container comprising the corresponding slurry formulation may be selected. The solution is receivable by a slurry generator. The solution or solution ingredients may be received by the slurry generator by any suitable method. For example, the solution may be poured from the container into the slurry generator. As another example, the container comprising the solution may be inserted into the slurry generator, for example via a cartridge or cassette.

Ice coefficient (defined as the percentage of ice in the slurry) is another property of the slurry. The ice coefficient is important because the slurry will be more effective in treating tissue when there are colder temperatures resulting from more ice in the slurry. However, if there is too much ice in the slurry, issues with flowability of the slurry arise due to clogging and blocking of flow, such as from agglomeration of ice particles. As an example, the ice coefficient may be about 2% to about 70% of the slurry by volume.

The ice coefficient should be high enough to provide enough ice to maintain an effective temperature of the slurry composition for treatment of the subcutaneous fat. However, the ice coefficient should be low enough to balance the effects of having too many ice particles in the slurry composition, such as blockage of the needle and flowability issues that arise from having too much ice present in the slurry composition.

In order to determine the cooling capacity of the slurry, the ice coefficient must be known. Melting ice is about 80 times more effective than a single degree of temperature change. When ice freezes, it will also raise the solute concentration in solution and therefore decrease the freezing point of the solution.

The ice coefficient may be measured by any suitable method. For example, calorimetry, conductivity, and temperature measurement methods may be used. As an example, calorimetry is a direct measurement of the cooling capacity of the solution, requiring no proxy to determine ice coefficient. For example, a known volume of slurry is added to a known volume of water at a known temperature. The amount of water temperature change is used to determine the cooling capacity of the slurry that was added. According to the solute concentration and the volume, the ice coefficient can then be accurately calculated. As another example, conductivity measurements function as a proxy for the ice-coefficient by monitoring the concentration of solutes in the liquid portion of the media. The increase in solute content that arises in the liquid from water being frozen increases the conductivity of the solution. This can then be monitored using a conductivity probe.

The volume of slurry produced by the generator is based on amount of solution provided to the generator. In some embodiments, containers of the invention comprising the solution may be standard containers comprising the same amount of solution, such as about 30 ml per container. In some embodiments, the containers may be produced in various sizes. For example, containers may be available based on a standard sizing system, such as a small size of about 30 ml, a medium size of about 90 ml, and a large size of about 330 ml. In certain embodiments, approximately about 1 ml to about 60 ml of slurry is used per injection. In some embodiments, one treatment area may be injected multiple times to total about 240 ml of slurry injected. Patients may be administered any suitable number of injections. For example, patients may have multiple injection sites and multiple injections.

Tonicity is another property of the slurry and is closely related to osmolality and osmolarity. Tonicity is the measure of an effective osmotic pressure gradient, or the measurement of osmotic pressure between two solutions. Osmolarity is the number of osmoles of solute per volume of solution (Osm/L), while osmolality is the number of osmoles of solute per mass of solvent (Osm/kg). Osmolarity and osmolality can be measured by any suitable method, such as by freezing point depression (FPD) and vapor point deficit (VPD). Injectable products generally are developed as isotonic solutions. A solution is isotonic when the solution has the same osmotic pressure as some other solution, for example having the same osmotic pressure as a cell or body fluid. When the osmotic pressure is lower than a particular fluid, the solution is hypotonic. Similarly, when the osmotic pressure is higher than a particular fluid, the solution is hypertonic. Osmolality and osmolarity are important when considering compositions and formulations for injection into patients, such as humans. If osmolality/osmolarity are too high, injury in the local area may result in tissue redness, blistering, tissue necrosis, and ulceration. Furthermore, hypertonicity-induced effects of subcutaneous administration include enhanced site irritation and pain, enhanced tissue permeability, and possible tissue damage. As such, the present invention tailors the osmolality to minimize inflammation effects (heat, redness, swelling, and pain) associated with injection or administration of the slurry.

To address the osmolarity and tonicity-related effects, the slurry may have an osmolality of less than about 2,200 milli-Osmoles/kg. In some embodiments, the slurry has an osmolality of less than about 600 milli-Osmoles/kg. The present invention provides a slurry in which the osmolality is well-tolerated by tissue.

Once the slurry is injected into a subject's body, the slurry operates to remove or reduce fat by causing cryolipolysis, or cell death by freezing of fat cells. Therefore, for this application, a temperature of the slurry should be cold enough to cause cell death. However, the temperature should be warm enough to avoid tissue redness, blistering, tissue necrosis, and ulceration. For example, the temperature may be about −25° C. to about 10° C. In some embodiments, the temperature is about −6° C. to about 0° C.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A system for generating slurry, the system comprising:
one or more containers, each container comprising one or more solution ingredients; and
a slurry generator for generating slurry from the one or more solution ingredients,
wherein the slurry comprises ice particles capable of flowing through a cannula.

2. The system of claim 1, wherein the one or more containers is inserted into the slurry generator.

3. The system of claim 1, wherein each of the one or more solution ingredients is contained in a separate container.

4. The system of claim 1, wherein each separate container is in fluid communication with the slurry generator.

5. The system of claim 1, wherein the slurry is produced by adjusting parameters of the slurry generator.

6. The system of claim 5, wherein the parameters are shown on a display of the slurry generator.

7. The system of claim 5, wherein the parameters comprise temperature, needle gauge, ice percentage by volume, and slurry generation time.

8. The system of claim 7, wherein the temperature comprises about −25° C. to about 10° C.

9. The system of claim 7, wherein the flow rate comprises about 20 ml/min to about 200 ml/min.

10. The system of claim 7, wherein the ice percentage by volume is about 2% to about 50%.

11. The system of claim 7, wherein the slurry generation time comprises less than about 10 minutes to about 10 hours.

12. The system of claim 1, wherein the slurry is configured to be introduced to a patient.

13. The system of claim 1, wherein the cannula is a needle.

14. The system of claim 13, wherein the needle has a gauge size from about 8G to about 25G.

15. The system of claim 1, wherein the slurry has an osmolality of less than about 2,200 milli-Osmoles/kilogram.

16. The system of claim 15, wherein the slurry has an osmolality of less than about 600 milli-Osmoles/kilogram.

17. The system of claim 1, wherein the slurry has a pH from about 4.5 to about 9.

18. The system of claim 1, wherein the ice particles have a size of less than about 1 mm.

19. The system of claim 18, wherein the size is less than about 0.25 mm.

20. The system of claim 1, wherein the solution ingredients comprise:
liquid water; and
one or more additives.

21. The system of claim 20, wherein the one or more additives are additives affecting flowability of the slurry.

22. The system of claim 20, wherein the one or more additives are additives affecting tonicity of the slurry.

23. The system of claim 20, wherein the one or more additives comprise at least one of sodium chloride, glycerol, sodium carboxymethylcellulose (CMC), dextrose, xanthan gum, polyethylene glycol, cellulose, polyvinyl alcohol, polyvinylpyrrolidone, guar gum, locust bean gum, carrageenan, alginic acid, gelatin, acacia, and carbopol.

24. A method of generating a slurry comprising:
providing one or more solution ingredients into to a slurry generator; and
adjusting parameters of the slurry generator, thereby generating slurry comprising ice particles capable of flowing through a cannula.

25. The method of claim 24, wherein the one or more solution ingredients are provided in one or more containers.

26. The method of claim 25, wherein the one or more containers is inserted into the slurry generator.

27. The method of claim 24, wherein each of the one or more solution ingredients is in a separate container.

28. The method of claim 27, wherein each separate container is in fluid communication with the slurry generator.

29. The method of claim 24, wherein the parameters are shown on an interactive display of the slurry generator.

30. The method of claim 24, wherein the parameters comprise temperature, needle gauge, ice percentage by volume, and slurry generation time.

31. The method of claim 30, wherein the temperature comprises about −25° C. to about 10° C.

32. The method of claim 30, wherein the flow rate comprises about 20 ml/min to about 200 ml/min.

33. The method of claim 30, wherein the ice percentage by volume comprises about 2% to about 50%.

34. The method of claim 30, wherein the slurry generation time comprises less than about 10 minutes to about 10 hours.

35. The method of claim 24, wherein the cannula is a needle.

36. The method of claim 35, wherein the needle has a gauge size from about 8G to about 25G.

37. The method of claim 24, wherein the slurry has an osmolality of less than about 2,200 milli-Osmoles/kilogram.

38. The method of claim 37, wherein the slurry has an osmolality of less than about 600 milli-Osmoles/kilogram.

39. The method of claim 24, wherein the slurry has a pH from about 4.5 to about 9.

40. The method of claim 24, wherein the ice particles have a size of less than about 1 mm.

41. The method of claim 40, wherein the size is less than about 0.25 mm.

42. The method of claim 24, wherein the solution ingredients comprise:
liquid water; and
one or more additives.

43. The method of claim 42, wherein the one or more additives are additives affecting flowability of the slurry.

44. The method of claim 42, wherein the one or more additives are additives affecting tonicity of the slurry.

45. The method of claim 42, wherein the one or more additives comprise at least one of sodium chloride, glycerol, sodium carboxymethylcellulose (CMC), dextrose, xanthan gum, polyethylene glycol, cellulose, polyvinyl alcohol, polyvinylpyrrolidone, guar gum, locust bean gum, carrageenan, alginic acid, gelatin, acacia, and carbopol.
